(19) United States
(12) Patent Application Publication
(10) Pub. No.: US 2021/0289778 A1
(43) Pub. Date: Sep. 23, 2021

(54) ANTIMICROBIAL NONWOVEN WET WIPE BONDED WITH A CATIONIC BINDER

(71) Applicant: WACKER CHEMIE AG, Munich (DE)

(72) Inventors: John BOYLAN, Bethlehem, PA (US); Dennis SAGL, Fogelsville, PA (US)

(73) Assignee: WACKER CHEMIE AG, Munich (DE)

(21) Appl. No.: 16/980,613

(22) PCT Filed: Dec. 14, 2018
(86) PCT No.: PCT/US2018/065730

§ 371 Date: Sep. 14, 2020

(51) Int. Cl. A01N 25/34; A01N 33/12; A01N 25/10; A01N 25/22

(52) U.S. Cl. CPC A01N 25/34; A01N 25/22; A01N 25/10; A01N 33/12

(57) ABSTRACT

An antimicrobial nonwoven wet wipe includes i) a fibrous nonwoven substrate bonded with a crosslinkable VAE dispersion stabilized with one or more cationic vinyl alcohol-N-vinyl amine copolymer protective colloids, and ii) an aqueous lotion containing a cationic disinfectants absorbed in the nonwoven substrate. No anionic surfactants are present. The antimicrobial nonwoven wet wipes are produced by a) applying an aqueous composition including a crosslinkable VAE dispersion stabilized with one or more cationic vinyl alcohol-N-vinyl amine copolymers to a nonwoven substrate; b) drying; and c) applying an aqueous composition to the product of step b). At least one of the first and second aqueous com-positions includes a cationic disinfectant.

### CROSS REFERENCE TO RELATED APPLICATIONS

This application is the U.S. National Phase of PCT Appln. No. PCT/EP2018/065730 filed Dec. 14, 2018, the disclosure of which is incorporated in its entirety by reference herein.

### BACKGROUND OF THE INVENTION

### 1. Field of the Invention

The invention relates to a nonwoven wet wipe comprising a nonwoven substrate and an aqueous antimicrobial lotion, in which the nonwoven substrate is bonded with a cationic binder.

### 2. Description of the Related Art

Self-crosslinking dispersion binders for the airlaid nonwovens industry are typically stabilized with some amount of anionic surfactant(s). U.S. Pat. No. 5,109,063 A discloses a process for making a vinyl acetate ethylene N-methylol acrylamide (NMA) copolymer emulsion for nonwoven binder applications. The emulsifying system consists of a salt of an alkyleneoxypoly(ethyleneoxy) sulfate. Binders of this type are commonly utilized in lotionized wet wipe applications where the lotion is compatible with the anionic nature of the self-crosslinking binder used to provide integrity to the nonwoven article.

However, if the lotion contains charged cationic compounds, as in the case of quarternary ammonium salt disinfectants, such as benzalkonium chloride, commonly referred to as quats, then the anionic nature of the surfactants tends to neutralize the efficacy of the quarternary disinfectant, diminishing the microbial killing power of the disinfectant. Therefore, the nonwoven article needs to be bound with either a nonionic or cationically stabilized dispersion. US 2004/0137815 A1 claims a nonwoven antimicrobial wipe comprising a fibrous nonwoven substrate which is bonded with a nonionic binder and which comprises a cationic disinfectant. US 2002/0183233 A1 discloses an improvement of antimicrobial nonwoven wipes by the addition of a salt (cationic release agent) to the lotion, which improves the release of the cationic disinfectant. The cationic release agent competes with the cationic quat for the anionic sites in the wipe substrate. A nonionic or cationic binder is not utilized. US 2005/0025668 A1 describes a wet wipe lotionized with a disinfectant. The nonwoven substrate is free of latex binder and an aqueous hypohalite releasing composition is applied to the substrate. US 2002/0031486 A1 discloses cleansing wipes composed of a water insoluble nonwoven substrate and an aqueous, antimicrobial cleansing composition comprising a cationic disinfectant and a nonionic surfactant. U.S. Pat. No. 5,326,809 describes poly(vinylalcohol)-co-(vinylamine) as protective colloids in aqueous emulsion polymerization. It does not disclose the application for antimicrobial nonwoven wet wipes.

There remains a need for a simple and cost-effective way for improving the efficacy of cationic disinfectants in wet wipe compositions.

### SUMMARY OF THE INVENTION

The invention provides an antimicrobial nonwoven wet wipe that includes i) a fibrous nonwoven substrate bonded with a cross-linkable vinyl acetate ethylene (VAE) dispersion stabilized with one or more cationic vinyl alcohol-N-vinyl amine copolymers and optionally one or more nonionic surfactants or nonionic protective colloids, and ii) absorbed in the nonwoven substrate, an aqueous lotion including one or more cationic disinfectants. No anionic surfactants are present in the antimicrobial nonwoven wet wipe.

The invention also provides a method for producing the antimicrobial nonwoven wet wipe, including a) applying a first aqueous composition including a crosslinkable VAE dispersion stabilized with one or more cationic protective colloids from the group of cationic vinyl alcohol-N-vinyl amine copolymers, and optionally one or more nonionic surfactants or nonionic protective colloids to a nonwoven substrate; b) drying the composition; and c) applying a second aqueous composition to the product of step b). At least one of the first and second aqueous compositions includes one or more cationic disinfectants.

### DETAILED DESCRIPTION OF THE PREFERRED EMBODIMENTS

The inventors have found that the release, and therefore the efficacy, of cationic disinfectants, for example quaternary ammonium disinfectants, is diminished if anionic surfactants are present in a nonwoven wet wipe. Anionic surfactants are commonly used to stabilize vinyl acetate ethylene copolymer (VAE) dispersions used as binders for nonwoven substrates. However, the inventors have found that VAE dispersions that are cationically stabilized with one or more cationic protective colloids from the group of vinyl alcohol-N-vinyl amine copolymers, provide improved efficacy of the cationic disinfect-ant in wet wipe compositions compared with anionically stabilized or nonionically stabilized VAE binder dispersions. The binder compositions according to the invention, and the wet wipes produced from them, are free, or essentially free, of anionic surfactants so as to avoid interfering with the activity of the cationic disinfectant.

Components and methods of making antimicrobial nonwoven wet wipes bonded with nonionic VAE binders according to the invention will now be discussed in detail. Unless the context indicates otherwise, percentages of materials recited herein are by weight.

VAE Copolymer

Unless specified otherwise, percentages by weight of monomers mentioned herein are based an the total weight of all monomers used for the polymerization to make the VAE copolymer, with the weight percentages of the monomers summing in each case to 100%. VAE copolymers for use as binders according to the invention comprise polymerized units of vinyl acetate, ethylene, an N-methylol-functional monomer, and (meth)acrylamide, i.e., acrylamide and/or methacrylamide. Vinyl acetate is copolymerized in general in an amount of at least 65% by weight, or at least 70%, and at most 94.5% by weight, or at most 85%. Ethylene is copolymerized in general in an amount of at least 5% by weight, or at least 10%, and at most 30% or at most 20% by weight.

The fraction of the N-methylol-functional monomer in the copolymer is typically at least 0.1% by weight, or at least 0.5%, 1%, or 2% by weight, and is typically at most 10.0% by weight, or at most 8%, or 5% by weight, based in each case an the total weight of monomers used for the polymerization. Suitable amounts of N-methylol-functional monomer, relative to the total of N-methylol-functional monomer plus (meth)acrylamide, are at least 25% by weight, or at least 30%, 40%, 45%, 50%, or 55% by weight. The amount will be at most 85%, or at most 80%, 75%, 70%, 65%, or 60% by weight. The total amount of N-methylol-functional monomer plus (meth)acrylamide present in the copolymer is at least 0.2% by weight, or at least 0.5%, 1%, 3%, or 5% by weight, and at most 5.0% by weight, or at most 8%, 10%, or 15% by weight.

Suitable exemplary N-methylol-functional monomers for making the copolymer include N-methylolacrylamide (NMA), N-methylolmethacrylamide, allyl N-methylolcarbamate, and esters of N-methylolacrylamide, N-methylol-methacrylamide, or of allyl N-methylolcarbamate. N-methylo-lacrylamide and N-methylol-methacrylamide are particularly preferred. The N-methylol-functional monomer is used in combination with acrylamide and/or methacrylamide, preferably in combination with acryla-mide. Most preferred are blends of N-methylolacrylamide and acryla-mide. Such blends are commercially available, for example being a 48% by weight aqueous solution of NMA and acrylamide in a 1:1 molar ra-tio, available under the tradename CYLINK® NMA-LF MONOMER (Cytec Industries, Woodland Park, N.J.), or an aqueous solution containing 28% by weight N-methylolacrylamide and 20% b.w. acrylamide, available under the tradename FLOCRYIJD NMA 2820 (SNF Floerger, Andrezieux, France). Alternatively, the NMA and acrylamide may be added separately to the polymerization feed.

In addition to NMA, other N—(C₁₋₄) alkylol (meth) acrylamides may be included in the VAE copolymer. Olefinically unsaturated monomers containing cellulose-reactive moieties may also be included, for example those containing aldehyde, protected aldehyde, and glycolic acid moieties. Examples include i-butoxymethylacrylamide, acrylamidoglycolic acid, acrylamidobutyraldehyde, and dialkyl acetals of acrylamidobutyraldehyde in which the alkyl groups each individually have 1 to 4 carbon atoms.

Optionally, the range of available properties for the copolymer in the dispersion may be extended by including additional monomers in the VAE copolymer. Typically, suitable comonomers are monomers with a single polymerizable olefinic group. Examples of such comonomers are vinyl esters of carboxylic acids having 3 to 18 C atoms. Preferred vinyl esters are vinyl propionate, vinyl butyrate, vinyl 2-ethylhexa-noate, vinyl laurate, 1-methyl vinyl acetate, vinyl pivalate, and vinyl esters of α-branched monocarboxylic acids having 9 to 11 C atoms, examples being VEOVA9™ or VEOVA10™ esters (available from Momentive Specialty Chemicals, Houston, Tex.). Other suitable comonomers include esters of acrylic acid or methacrylic acid with unbranched or branched alcohols having 1 to 15 C atoms. Exemplary methacrylic esters or acrylic esters include methyl acrylate, methyl methacrylate, ethyl acrylate, ethyl methacrylate, propyl acrylate, propyl methacrylate, n-butyl acrylate, n-butyl methacrylate, 2-ethylhexyl acrylate and norbornyl acrylate. Other suitable comonomers include vinyl halides such as vinyl chloride, or olefins such as propylene. In general, the further comonomers are copolymerized in an amount of 0.5 to 30% by weight, preferably 0.5 to 20% by weight, based on the total weight of monomers used for the polymerization.

Optionally, 0.05 to 10% by weight, based on the total weight of mon-omers used for the polymerization, of other monomers (auxiliary mono-mers) may additionally be copolymerized in forming the dispersion. Auxiliary monomers include a polymerizable olefinic group and at least one additional functional group. Examples of auxiliary monomers include acrylonitrile and diesters of fumaric acid and maleic acid, for example the diethyl and diisopropyl esters. Typically, there is only one polymerizable olefinic group in each monomer used to make the VAE copolymer, although in some cases there may be more.

On the other hand, ethylenically unsaturated monomers that contain carboxylic acid, sulphonic acid, or phosphate or phosphonate acid groups, salts of these, or groups that hydrolyze to these when used to make wet wipes according to the invention, are typically excluded from VAE copolymers used as binders to make the wipes. Specific examples include acrylic acid, methacrylic acid, fumaric acid, maleic acid, maleic anhydride, vinylsulphonic acid, and 2-acrylamido-2-methyl-propanesulphonic acid.

The choice of monomers or the choice of the proportions by weight of the monomers is preferably made in such a way that, in general, the copolymer has a suitable glass transition temperature (Tg). Typically, the Tg is at least −10° C., or at least −5° C., or at least 0° C., and at most +20° C., or at most +15° C., or at most +10° C. The glass transition temperature Tg of the copolymers can be determined in a known way by means of differential scanning calorimetry (DSC) with a heating rate of 10° C. per minute according to ASTM D3418-82 as onset temperature. The Tg can also be calculated approximately beforehand by means of the Fox equation. According to Fox T. G., Bull. Am. Physics Soc. 1, 3, page 123 (1956): 1/Tg=x1/Tg1+x2/Tg2+ . . . +xn/Tgn, where xn is the mass fraction (% by weight/100) of the monomer n and Tgn is the glass transition temperature in Kelvin of the homopolymer of the monomer n. Tg values for homopolymers are given in the Polymer Handbook 2nd Edition, J. Wiley & Sons, New York (1975).

Cationic Protective Colloid

Suitable cationic protective colloids, are cationic vinyl alcohol-N-vinyl amine copolymers, also referred to herein as cationic poly(vinyl alcohol)-co-(vinylamine). Vinyl alcohol-N-vinyl amine copolymers are commercially available from Sekisui. The synthesis of these co-polymers is well known in the art and for example described in EP 0339371 A2 or in WO 2006/082157 A1. In the first step of preparation vinyl acetate and N-vinyl formamide are copolymerized to yield a vinyl acetate-N-vinylformamide copolymer. In the second step the vinyl acetate-N-vinylformamide copolymer is saponified with acid or base catalysis to the vinyl alcohol-N-vinyl amine copolymer. In a third step, the vinyl alcohol N-vinylformamide copolymer is hydrolyzed with acid or base catalysis to vinyl alcohol N-vinylformamide copolymer.

After hydrolysis the vinyl alcohol-N-vinyl amine copolymer is preferably composed of 0 to 15 mole % vinyl acetate units, 50 to 99 mole % vinyl alcohol units, 0 to 10 mole % vinyl formamide units and 1 to 25 mole % vinyl amine units, and the data is in mole % summing in each case to 100 mole %. In general, the vinyl alcohol-N-vinyl amine copolymer has a weight average molecular weight Mw of 10,000 to 200,000, preferably Mw is 15,000 to 130,000. In general, the vinyl alcohol-N-vinyl amine copolymer has a viscosity, in 4% strength aqueous solution, of 1 to 30 mPas (Hoeppler viscosity, determined at 20° C. in accordance with DIN 53015). In general, the cationic charge of the vinyl alcohol-N-vinyl amine copolymers is introduced during emulsion polymerization of the VAE copolymer, which is in general performed at a pH below 6.

Additionally, one or more nonionic protective colloid(s) can be used to stabilize the VAE dispersions during and after the emulsion polymerization reaction. Suitable nonionic protective colloids include polyvinyl alcohol (PVOH) and nonionic cellulose derivatives, for example hydroxyethylcellulose. Other examples include polyvinylpyrrolidone, PVOH bearing ethylene oxide or polyethylene oxide substituents, and acetoacetylated PVOH. In addition, copolymers of PVOH may be used. Examples include ethylene and/or N-vinylpyrrolidone copolymers of vinyl alcohol. Polyvinyl alcohols are particularly useful. Suitable PVOH's include partially hydrolyzed polyvinyl alcohols hav-ing a degree of hydrolysis of 80 to 99 mol %, preferably 85 to 99 mol %, and a viscosity, in 4% strength aqueous solution, of 1 to 30 mPas, preferably 3 to 6 mPas (Hoeppler viscosity, determined at 20° C. in accordance with DIN 53015). Such PVOH's are commercially available or obtainable by processes known to the skilled person.

The cationic vinyl alcohol-N-vinyl amine copolymer, and optionally additional protective colloids, for example polyvinyl alcohol(s), will typically be present at a level of at least 0.1% by weight, or at least 0.2% or 0.5%. Typically, the level will be at most 10% by weight, or at most 5% or 1%. These percentages indicate the amount of protective colloid(s) relative to the total weight of all monomers used for the polymerization.

In addition to the cationic vinyl alcohol-N-vinyl amine copolymer, optionally nonionic surfactants (emulsifiers) can be present in the cross-linkable VAE dispersion. Preferred nonionic surfactants are ethoxylated branched or unbranched aliphatic alcohols, particularly having a degree of ethoxylation of 3 to 80 ethylene oxide units and C₅ to C₃₆ alkyl radicals. Preferred nonionic surfactants also include C₁₃-C₁₅ oxo-process alcohol ethoxylates having a degree of ethoxylation of 3 to 30 ethylene oxide units, and C₁₆-C₁₈ aliphatic alcohol ethoxylates having a degree of ethoxylation of 11 to 80 ethylene oxide units. Particularly preferred are C₁₂-C₁₄ aliphatic alcohol ethoxylates having a degree of ethoxylation of 3 to 30 ethylene oxide units, and copolymers of ethylene oxide and propylene oxide with a minimum content of at least 10% by weight of ethylene oxide. Preferably, these surfactants do not contain alkyl phenol ethoxylate structures. The total amount of surfactant is typically in a range from 0.5 to 5% by weight, preferably 1 to 3% by weight, based in each case on the total weight of the monomers.

Emulsion Polymerization Procedure

During polymerization of the VAE copolymer the polymerization mixture is stabilized with one or more vinyl alcohol-N-vinyl amine copolymers and optionally one or more nonionic surfactants or nonionic protective colloids. The VAE dispersions may be prepared by emulsion polymerization, typically at a temperature in a range from 40° C. to 100° C., more typically 50° C. to 90° C. and most typically 60° C. to 80° C. The polymerization pressure is generally between 10 and 100 bar, more typically between 25 and 90 bar, and may vary particularly between 45 and 85 bar, depending on the ethylene feed. The pH of the polymerization mixture is adjusted to pH<5, preferably the pH is adjusted to 3 to 5.

Polymerization may be initiated using a redox initiator combination 20 such as is customary for emulsion polymerization. Redox Initiator systems may be used to prepare VAE dispersions suitable for use according to the invention. The initiators may be formaldehyde-generating redox initiation systems such as sodium formaldehyde sulfoxylate. In some embodiments, however, it is desirable to minimize the formaldehyde level in the dispersion. In such cases, it is desirable to use a non-formaldehyde generating redox initiation system. In general, suitable non-formaldehyde generating reducing agents for redox pairs include, as non-limiting examples, those based on ascorbic, bisulfite, erythorbate or tartaric chemistries as known in the art, and a commercial reducing agent known as BRUGGOLITE® FF6M manufactured by Bruggeman Chemical of Heilbronn, Germany. Non-redox initiators may also be used, such as peroxides and azo-type initiators, all of which are well known in the art.

All of the monomers may form an initial charge, or all of the monomers may form a feed, or portions of the monomers may form an initial charge and the remainder may form a feed after the polymerization has been initiated. The feeds may be separate (spatially and chronologically), or all or some of the components may be fed after pre-emulsification. Once the polymerization process has ended, post-polymerization may be carried out using known methods to remove residual monomer, one example of a suitable method being post-polymerization initiated by a redox catalyst. Volatile residual monomers may also be removed by distillation, preferably at subatmospheric pressure, and, where appropriate, by passing inert entraining gases, such as air, nitrogen, or water vapor through or over the material.

The solids contents of suitable VAE copolymer dispersions as made are typically in a range from 45 to 75% by weight. The dispersions typically have a viscosity, if diluted to a 25% solids level, of at least 5 mPas, or at least 10, 20, or 30 mPas. The viscosity will typically be at most 800 mPas, or at most 700, 600, or 500 mPas. The viscosities are determined using a Brookfield Viscometer Model LVD with a #3 spindle at 60 rpm and 25° C.

Fibrous Nonwoven Substrate

The fibrous nonwoven substrate can be a natural fiber such as (but not limited to) cellulose fiber or wood pulp, or a synthetic fiber including but not limited to one or more of polyester, polyethylene, polypropylene and polyvinyl alcohol, or viscose fiber, or a combination of any of these, processed by a dry (airlaid, carded, rando) or wet laid process. The basis weight of the fibrous nonwoven substrate prior to treatment with the cationic binder composition is typically at least 10 g/m², or at least 45 g/m², and is typically at most 150 g/m², or at most 120 g/m².

Aqueous Disinfectant Lotion

The aqueous lotion that is absorbed in the bonded nonwoven substrate includes one or more cationic disinfectants. These are typically quaternary ammonium disinfectant compounds. Benzalkonium chloride is one specific example, although any other cationic disinfectant known in the art may be used instead or in addition. Some of the cationic disinfectant may be dissolved in the aqueous phase of the lotion while some is adsorbed on the surface of the fibers of the nonwoven substrate. Preferably, the cationic disinfectant(s) include(s) only one cationic moiety per molecule.

The aqueous lotion may optionally also contain salts that are not cationic disinfectant(s). Salts of any kind may be included, for example organic salts, inorganic salts, and salts comprising an organic anion and a metal, a non-disinfectant quaternary ammonium cation, or a nonquaternary ammonium cation, i.e., NH₄⁺ or a protonated primary, secondary, or tertiary amine. Nonlimiting examples include acetates, acetylides, ammonium salts (excluding quats), arsenates, astatides, azides, bihalide salts, bicarbonates, bisulfides, borides, borohydrides, borohalides, carbonates, citrates, cyanates, cyanides, formates, germanates, glycinates, palates, halides, hydrides, hydroselenides, hydrosulphides, hydroxides, imides, metaniobates, metaantalates, metavanadates, nitrates, nitrides, nitrites, oxides, perchlorates, phosphates, phosphonium salts, selenides, selenites, selenates, sulphides, sulphates, ternary salts, non-disinfectant tetraalkyl ammonium salts, tellurides, thiocyanates, and/or vanadates. Specific examples include potassium citrate, sodium citrate, sodium tartrate, potassium tartrate, potassium lactate, sodium lactate, salicylate salts of sodium and/or potassium, magnesium sulphate, sodium chloride, ammonium chloride, and/or potassium chloride.

However, any one or more of the abovementioned salts, or all salts other than cationic disinfectants, may be excluded.

The aqueous lotion may also comprise an organic solvent which, if present, will typically constitute at most 10% by weight of the lotion composition, or at most 5, 2, or 1% by weight. Examples include C₁₋₆ alkanols, C₁₋₆ diols, C₁₋₁₀ alkyl ethers of alkylene glycols, C₃₋₂₄ alkylene glycol ethers, and/or polyalkylene glycols. Specific types of solvents include alkanols such as methanol, ethanol, n-propanol, isopropanol, butanol, pentanol, and/or hexanol, and their various positional isomers; acetone; and glycol ethers such as ethylene glycol monopropyl ether, ethylene glycol monobutyl ether, ethylene glycol monohexyl ether, propylene glycol n-propyl ether, propylene glycol monobutyl ether, propylene glycol t-butyl ether, diethylene glycol monoethyl or monopropyl or monobutyl ether, di- or tripolypropylene glycol methyl or ethyl or propyl or butyl ether, acetate and/or propionate esters of glycol ethers. However, any one or more of the abovementioned solvents, or all solvents, may be excluded.

Making the Antimicrobial Nonwoven Wet Wipe

The cationic VAE binder composition is typically applied to the nonwoven substrate via spray application, saturation, gravure printing or foaming. The binder composition may optionally include a catalyst, for example an acidic compound or ammonium salt thereof. One example is ammonium chloride.

A wetting additive can be included in the binder composition to aid in the wetting not only of the formulated binder on the substrate, but also of the subsequent finished fibrous nonwoven substrate. The wetting additive should be either a nonionic or cationic type wetting surfactant so as not to reduce the efficacy of the cationic disinfectant added as a lotion to the bound nonwoven substrate. One example is SURFYNOL® 465, a nonionic ethoxylated acetylenic diol sold by Air Products. The wetting agent can be included in the binder composition at a level of 0.1 to 3 dry parts, based on the weight of dry polymer, but is more typically formulated at between 0.5 and 2 parts.

The composition is typically applied at a solids level between 0.5 to 30% by weight, depending on the desired loading on the substrate. Typically, the amount of binder on a dry basis will be at least 5% by weight, or at least 10% or 15%, based on the weight of the untreated substrate. It will typically be at most 50% by weight, or at most 40% or 30%.

After the binder composition is applied to the substrate, the substrate is dried. This is typically done at a temperature in a range from 120° C. to 160° C., but higher or lower temperatures may be used. After that, the aqueous lotion containing the cationic disinfectant may be applied.

Alternatively, the cationic disinfectant may be included in the VAE binder composition instead of being added separately in the lotion as described above. In that case, water and any other lotion components, for example solvents, can be added to the substrate after the binder composition has been applied and dried, and it is understood that some or all of the cationic disinfectant may dissolve in the water. Or, both modes of adding the cationic disinfectant may be used. In any of these modes, the aqueous lotion will typically be present in an amount of at least 50 parts lotion (wet basis), or at least 150, 200, or 250 parts, per 100 parts bonded substrate (dry basis). The amount of lotion will typically be at most 500 parts, or at most 400 or 350 parts, per 100 parts substrate. In all cases, the resulting wet wipe may be packaged in any way that is effective to minimize or avoid drying out.

### EXAMPLES

Measurement of Tg

The glass transition temperature (Tg) of the copolymers was determined by means of differential scanning calorimetry (DSC) using a Mettler-Toledo DSC1 dynamic differential scanning calorimeter with a heating rate of 10° C. per minute according to ASTM D3418-82 as onset temperature. The onset of the glass transition was evaluated in the 2nd heating cycle.

Measurement of Viscosity—Brookfield

Unless otherwise noted, Brookfield viscosities of copolymer dispersions and adhesive compositions were determined using a Brookfield Viscometer Model LVD with a #3 spindle at 60 rpm and 25° C. Viscosities of polyvinyl alcohols are Hoeppler viscosities of 4% by weight aqueous solutions, determined at 20° C. in accordance with DIN 53015.

The following vinyl acetate ethylene dispersions copolymerized with NMA and acrylamide were prepared and tested:

Comparison Example C1:

The following ingredients were mixed together: 3.200 kg of a 10% aqueous solution of SELVOL® 107 (a poly(vinyl alcohol) with average hydrolysis level of 98-99 mol %, 4% b.w. aqueous solution viscosity of 5.5 to 6.5 mPa·s, available from Sekisui), 3.200 kg of RHODASURF® TLA 3040 (a 40% b.w. solution of a tridecyl alcohol ethoxylate surfactant that has approximately 30 ethylene oxide units per tridecyl alcohol, available from Solvay), 1.600 kg of PLURONIC® F68 (an ethylene oxide/propylene oxide block copolymer, available from BASF Chemical Corp.), 0.640 kg of PLURONIC® L64 (an ethylene oxide/propylene oxide block copolymer, available from BASF Chemical Corp.) and 40.0 g of dibasic ammonium phosphate were dissolved in 27.100 kg of deionized water. The pH of this mixture was adjusted to 3.8 using 40.0 g of phosphoric acid (85%) and 1.7 g of ferrous ammonium sulfate was then added to the mixture. This mixture was added to a thirty-five gallon (132.5 l) pressure reactor that had been purged with nitrogen, and 54.372 kg of vinyl acetate was added with agitation (350 rpm). The reactor was purged with ethylene, the agitation was maintained at 350 rpm, and 9.595 kg of ethylene was added to the reactor. The temperature was then increased to 35° C., and 118.0 g of a 4.8% b.w. aqueous sodium erythorbate solution (pH adjusted to 4.5 with 85% phosphoric acid) was added to the reactor. The reactor contents were allowed to equilibrate and the pressure at this point was 392 psi (27 bar). A 4.0% b.w. aqueous solution of tert-butyl hydroperoxide and a 4.8% b.w. aqueous solution of sodium erythorbate (pH adjusted to 4.5 with 85% phosphoric acid) were continuously fed to the reactor at a rate of 10.0 g/min and 16.7 g/min respectively. After the temperature rose 1° C., the reactor temperature was allowed to increase to 75° C. over 60 minutes. In addition, 11.440 kg of an aqueous solution of NMA-LF (Flocryl® NMA 2820, an aqueous mix of approximately 28% b.w. N-methylolacrylamide and 20% b.w. acrylamide, available from SNF Floerger) was fed to the reactor over 180 minutes, and the feed line for the NMA-LF was rinsed to the reactor with an additional 0.375 kg of water. The addition rate for the NMA-LF was approximately constant over this 3-hour delay period.

The flow rates of the tert-butyl hydroperoxide and sodium erythorbate feeds were maintained at an approximately 1:1.7 ratio and the flows were adjusted so that the 75° C. reaction temperature was maintained. The unreacted vinyl acetate was measured during the course of the re-action and found to be 46.6% after 1 h, 26.1% after 2 h, 7.6% after 3 h, and 4.7% after 3.2 h. At the end of 3.5 h, the tert-butyl hydroperoxide and sodium erythorbate feeds were stopped, the reaction was cooled to 50° C. and the reaction mixture was transferred to a de-gasser to remove unreacted ethylene. The reactor was rinsed with 2.300 kg of water, which was also transferred to the degasser, and a mixture of 35.0 g of RHODOLINE® DF540 defoamer (available from Solvay) and 110.0 g of water were added to inhibit foam formation. In order to reduce unreacted vinyl acetate monomer below 0.1% b.w., 0.740 kg of an 8.0% b.w. aqueous sodium erythorbate solution and 0.740 kg of a 6.30% b.w. aqueous tert-butyl hydroperoxide solution were added over 40 minutes. Finally, 18.9 g of dodecylguanidine hydrochloride dissolved in 215 g of a 7.01% b.w. aqueous hydrogen peroxide solution was added over 30 minutes.

Solids content, viscosity, and Tg are listed in Table 1.

### Example 2

The following ingredients were mixed together: 811.5 g of a 10% aqueous solution of poly(vinyl alcohol)-co-(vinylamine) (88:12 vinyl alcohol:vinylamine molar ratio, with a 4 to 6 mPa·s viscosity for a 4% b.w. aqueous solution) and 100.0 g of deionized water. The pH of this mixture was adjusted to 3.5 using 33.8 g of a 50% b.w. aqueous solution of phosphoric acid, and 2.1 g of a 5% b.w. aqueous solution of ferrous ammonium sulfate was then added to the mixture. This mixture was added to a one gallon (3.8 l) stainless steel pressure reactor that had been purged with nitrogen, and 1100 g of vinyl acetate was added with agitation (200 rpm).

The reactor was purged with ethylene, the agitation was increased to 1000 rpm, and 245 g of ethylene was added to the reactor. The temperature was then increased to 32° C., and 7.3 g of a 4.75% b.w. aqueous sodium erythorbate solution (pH adjusted to 4.5 with 50% phosphoric acid) was added to the reactor. An aqueous solution of 1.50% b.w. hydrogen peroxide and a 4.75% b.w. aqueous solution of sodium erythorbate were each continuously fed to the reactor at a rate of 0.2 g/min. After the temperature rose 1° C., the reactor temperature was allowed to increase to 85° C. over 80 minutes.

The hydrogen peroxide and sodium erythorbate feeds were maintained at equal flow rates and adjusted so that the 85° C. reaction temperature was maintained. The unreacted vinyl acetate was measured during the course of the reaction and found to be 23.5% after 0.5 h, 9.2% after 1.5 h, and 1.0% after 2.5 h. At the end of 2.5 h, the hydrogen peroxide and sodium erythorbate feeds were stopped, the reaction was cooled to 60° C. and the reaction mixture was transferred to a degasser to remove unreacted ethylene. A mixture of 1.0 g of Foamaster VF defoamer (available from Solvay) and 5 g of water were added to inhibit foam formation. In order to reduce unreacted vinyl acetate monomer below 0.1% b.w., 25.0 g of an 8% b.w. aqueous sodium erythorbate solution and 20.0 g of an 8.0% b.w. aqueous tert-butyl hydroperoxide solution were added over 15 minutes while the temperature was maintained at or above 30° C.

Solids content, viscosity, and Tg are listed in Table 1.

### Example 3

The following ingredients were mixed together: 33.895 kg of a 10% b.w. aqueous solution of poly(vinyl alcohol)-co-(vinylamine) (88:12 vinyl alcohol:vinylamine molar ratio, with a 4 to 6 mPa·s viscosity for a 4% b.w. aqueous solution) and 1.959 kg of deionized water. The pH of this mixture was adjusted to 4.0 using 834 g of phosphoric acid (85%), and 4.4 g of ferrous ammonium sulfate was then added to the mixture. This mixture was added to a thirty-five gallon (132.5 l) pressure reactor that had been purged with nitrogen, 2.300 kg of deionized water was used to rinse this initial charge into the reactor, and 45.948 kg of vinyl acetate was added with agitation (375 rpm).

The reactor was purged with ethylene, the agitation was maintained at 375 rpm, and 10.235 kg of ethylene was added to the reactor. The temperature was then increased to 32° C., and 246.0 g of a 2.9% b.w. aqueous sodium erythorbate solution (pH adjusted to 4.5 with 85% b.w. phosphoric acid) was added to the reactor. The reactor contents were allowed to equilibrate. A 1.5% b.w. aqueous solution of hydrogen peroxide and a 2.9% b.w. aqueous solution of sodium erythorbate (pH adjusted to 4.5 with 85% b.w. phosphoric acid) were continuously fed to the reactor at a rate of 6.7 g/min and 6.8 g/min respectively. After the temperature rose 0.5° C., the reactor temperature was allowed to increase to 77° C. over 90 minutes. In addition, 5.367 kg of a 7.63% b.w. active aqueous solution of NMA-LF (Flocryl NMA 2820, an aqueous mix of approximately 28% b.w. N-methylolacrylamide and 20% b.w. acrylamide, available from SNF Floerger) was fed to the reactor over 180 minutes, and the feed line for the NMA-LF was rinsed to the reactor with an additional 0.375 kg of deionized water. The addition rate for the NMA-LF was approximately constant over this 3-hour delay period.

The flow rates of the hydrogen peroxide and sodium erythorbate feeds were maintained at an approximately 1:1 ratio and the flows were adjusted so that the 77° C. reaction temperature was maintained. The unreacted vinyl acetate was measured during the course of the reaction and found to be 36.7% after 1 h, 18.4% after 2 h, 7.5% after 3 h, and 2.0% after 4 h. At the end of 4.0 h, the hydrogen peroxide and sodium erythorbate feeds were stopped, the reaction was cooled to 50° C. and the reaction mixture was transferred to a degasser to remove unreacted ethylene. The reactor was rinsed with 2.300 kg of deionized water, which was also transferred to the degasser, and a mixture of 63.0 g of Foamaster M02185 defoamer (available from BASF) and 415.0 g of water were added to inhibit foam formation. In order to reduce unreacted vinyl acetate monomer below 0.1% b.w., 1.044 kg of an 8.0% b.w. aqueous sodium erythorbate solution and 0.836 kg of an 8.0% b.w. aqueous tert-butyl hydroperoxide solution were added over 45 minutes.

Finally, 0.609 kg of a 7.0% b.w. aqueous hydrogen peroxide solution was added over 30 minutes.

Solids content, viscosity, and Tg are listed in Table 1.

Testing and Results:

Testing and measurements to demonstrate the improvement in cationic disinfectant efficacy within nonwoven substrates followed these steps:

1. Application of Binder to Nonwoven:

The nonwoven base substrates used in the study were produced via the airlaid process and were made of 88% b.w. cellulose fiber and 12% b.w. synthetic bi-component fiber comprised of a polyethylene sheath and a polyester core. Basis weight of the base airlaid was about 90 grams/m². The binders listed in Table 1 were formulated as shown in Table 2 and sprayed onto both sides of the airlaid substrate as a 20% b.w. solids aqueous composition to obtain a polymer add-on of 20% b.w. (dry an dry substrate) and dried for 3 minutes at 150° C. in a Mathis through air dryer. The bound substrates were placed in a constant temperature and humidity room at 70 F (21° C.) and 50% relative humidity prior to the application of the quaternary amine. The physical properties of these substrates bound with the binders listed are shown in Table 3. Dry and wet tensile breaking strength testing were performed according to ASTM method D 5035-95. Quaternary Amine application to nonwoven substrate:

The nonwoven substrates foiled in Step 1 above were wetted with a 378 ppm benzalkonium chloride (a quaternary amine) aqueous solution at a level of 300%, wet benzalkonium chloride solution to dry weight of the nonwoven substrate. The treated substrates were then placed in a plastic bag and sealed to prevent evaporation. The treated substrates sealed in the bags were allowed to sit for 42 hours, after which the benzalkonium chloride solution was extracted from the wipes into individual brown bottles, according to the binder used and each bottle was then capped.

2. Benzalkonium Chloride Analysis:

The benzalkonium chloride solutions extracted from the treated wipes were diluted at a 1:10 ratio with deionized water further purified with a commercially available Milli-Q® water purifier. A benzalkonium chloride standard from Sigma-Aldrich was used to prepare aqueous standards. Standard solutions ranging from 0.5 ppm to 45 ppm in concentration were used to create a four-point external linear calibration curve. The square of the correlation coefficient (R²) was 0.999905. The diluted test benzalkonium chloride solutions were then subjected to high pressure liquid chromatography (HPLC) separation was achieved with a Waters Alliance system and a Surfactant Plus column using an acetonitrile/potassium phosphate mobile phase.

A Waters PDA detector was used for the peak detection. Area under the peak indicates the amount of benzalkonium chloride present. Results (Benzalkonium Chloride Efficacy in ppm) from the analysis are shown in Table 1.

4. Physical Strength Measurements of the Bonded Airlaid Substrates:

The cross direction (CD) wet and dry tensile breaking strength of the treated airlaid nonwoven substrates was measured an an Instron tensile tester using ASTM method D 5035-95. The bonded substrate was die cut using a 5.1 cm×25.4 cm (2 inches×10 inches) die cutter to prepare samples for tensile strength determination. The strips were placed in the jaws of the Instron mechanical tensile tester. For dry tensile determination the die cut samples were placed vertically into the jaws of the tester and the test was initiated. The tensile tester provides the statistics of the maximum tensile achieved at break. A cross head speed of 15.2 cm/minute (6 inches/minute) was used and a gauge length of 20.3 cm (8 inches) was set for dry tensile determination. A number of tests were performed, and the average calculated and reported.

Wet tensile measurement was determined similarly except that the sample was placed into a Finch Cup apparatus that included a water-filled reservoir. The sample was looped around a metal bar and then dipped into the water and held there for 15 seconds. The tensile test was then initiated. A gauge length of 5.1 cm (2 inches) was used due to the loop effect of the tensile strip. The maximum wet strength was determined by the tensile tester. Several tests were performed, and the average was calculated.

The results of the physical strength measurements of the bonded air-laid substrates are shown in Table 3.

TABLE 1
Quaternary Amine Efficacy Testing and Results

| | | | | | | Benzalkonium |
|---|---|---|---|---|---|---|
| | | | | | | Chloride Efficacy |
| | | | | | | as a % of Mother |
| Samp1e | Stabilizer | Solids | pH | Viscosity | Tg | Solution |
| Vinnapas | anionic | 52.3% | 5.3 | 94 mPa · s | 8.6° C. | 5.2% |
| Comp. Ex. C1 | nonionic | 56.1% | 5.4 | 340 mPa · s | 1.9° C. | 31.4% |
| Example 2 | cationic | 53.1% | 4.1 | 330 mPa · s | 5.5° C. | 77.3% |
| Example 3 | Cationic | 55.5% | 3.9 | 624 mPa · s | 8.4° C. | 49.1% |

VINNAPAS® 192 of Wacker Chemie AG is a commercially available self-crosslinking VAE dispersion with 4.8% b.w. of a 1:1 molar blend of NMA and acrylamide, stabilized with 2.5% b.w., relative to polymer, of an anionic surfactant system and a solids content of the dispersion of 52.3%.

The test results for the efficacy of the benzalkonium chloride disinfectant in the wet wipe compositions show a two to three times higher efficacy of the cationic binder in comparison with nonionic binder, and a more than 10 times higher efficacy compared to anionic bonded substrates.

TABLE 2
Binder Formulation

| Component | Dry Parts |
|---|---|
| VAE | 100 |
| Ammonium chloride | 0/1 |
| SURFYNOL ® 465 Wetting | 1 |

TABLE 3
Cross direction (CD) Wet and Dry Tensile Breaking Strength

| | | | CD Dry | CD Wet |
|---|---|---|---|---|
| | | NMA-LF an | tensile gram | Tensile gram |
| Sample | Stabilizer | VAE % | force/5 cm | force/5 cm |
| Vinnapas 192 | Anionic | 4.8 | 3591 | 1518 |
| Control for | | | | |
| Comp. Ex. | | | | |
| Comp. Ex. 1 | Nonionc | 4.8 | 3151 | 1338 |
| Vinnapas 192 | Anionic | 4.8 | 3848 | 1688 |
| Control for | | | | |
| Ex. 2 | | | | |
| Example 2 | Cationic | 0.0 | 3573 | 1400 |
| Vinnapas 192 | Anionic | 4.8 | 3909 | 1730 |
| Control for | | | | |
| Ex. 3 | | | | |
| Example 3 | Cationic | 0.6 | 3716 | 1581 |

The comparative dispersion samples shown in Table 3 were run as separate experiments using Vinnapas® as the control comparison. Separate experiments were conducted due to the variation in the base airlaid substrate and normal experimental error. These variations result in differences in the substrate tensile value bound with the Vinnapas® 192 between each experimental run. Accordingly, it is important to show how each of the examples compares to its respective Vinnapas® 192 control.

The antimicrobial nonwovens bonded with the cationic binder of examples 2 and 3 show a mechanical strength similar to the anionic or nonionic bonded nonwovens, even if no crosslinkable NMA is copolymerized (Example 2) or even if only one-eighth of NMA is copolymerized (Example 3). From the experimental data embodiments made according to Example 2 having an add-on of between 1.0% and 25.0% of dry dispersion on the dry nonwoven provides a dry nonwoven tensile strength of between 250 grams force/5 cm width and 5000 grams/5 cm width, and a wet tensile strength of between 250 grams force/5 cm width and 3000 grams/force/5 cm width. Embodiments made according to Example 3 having an add-on of between 1.0% and 25.0% of dry dispersion on the dry nonwoven provides a dry nonwoven tensile strength of between 250 grams force/5 cm width and 5000 grams/5 cm width, and a wet tensile strength of between 250 grams force/5 cm width and 3000 grams/force/5 cm width. In consequence, the much lower NMA content provides a drastic reduction of the formaldehyde emission.

Absorbance rate of substrate bound with cationic binder, although slower would not hinder the total absorption of fluid, (such as lotions used in antimicrobial application) into the substrate.

1.-10. (canceled)

11. An antimicrobial nonwoven wet wipe, comprising:
i) a fibrous nonwoven substrate bonded with a cross-linkable VAE dispersion stabilized with one or more cationic vinyl alcohol-N-vinyl amine copolymer protective colloids, and
ii) absorbed in the nonwoven substrate, an aqueous lotion comprising one or more cationic disinfectants,
wherein no anionic surfactants are present in the antimicrobial nonwoven wet wipe.

12. The antimicrobial nonwoven wet wipe of claim 11,
wherein the cross-linkable VAE dispersion is additionally stabilized with one or more nonionic protective colloid(s).

13. The antimicrobial nonwoven wet wipe of claim 11,
wherein the cross-linkable VAE dispersion is additionally stabilized with one or more nonionic surfactants.

14. The antimicrobial nonwoven wet wipe of claim 11, wherein the vinyl alcohol-N-vinyl amine copolymers comprise 50 to 99 mole % vinyl alcohol units, 0 to 10 mole % vinyl formamide units and 1 to 25 mole % vinyl amine units.

15. The antimicrobial nonwoven wet wipe of claim 11, wherein the one or more cationic disinfectants include a quaternary ammonium disinfectant.

16. The antimicrobial nonwoven wet wipe of claim 11, wherein the one or more cationic disinfectants include benzalkonium chloride.

17. A method for producing an antimicrobial nonwoven wet wipe of claim 11, comprising:
a) applying a first aqueous composition comprising a crosslinkable VAE dispersion stabilized with one or more cationic protective col-loids out of the group of cationic vinyl alcohol-N-vinyl amine copolymer protective colloids to a nonwoven substrate;
b) drying the composition; and
c) applying a second aqueous composition to the product of step b);
wherein at least one of the first and/or second aqueous compositions comprises one or more cationic disinfectants.

18. The method of claim 17, wherein the first aqueous com-position comprises one or more of said cationic disinfectants.

19. The method according to claim 17, wherein the second aqueous composition comprises one or more of said cationic disinfectants.

20. The method of claim 17, wherein the antimicrobial nonwoven wet wipe is bound with the cationic vinyl alcohol-N-vinyl amine copolymer-stabilized vinyl acetate-ethylene dispersion at a dry add-on of between 1.0% and 25% on the dry nonwoven, providing a dry nonwoven tensile strength of between 250 grams force/5 cm width and 5000 grams/5 cm width and a wet tensile strength of between 250 grams/5 cm width and 3000 grams/5 cm width as measured on an Instron tensile tester using ASTM method D 5035-95.
